(19) United States
(12) Patent Application Publication
(10) Pub. No.: US 2004/0161847 A1
(43) Pub. Date: Aug. 19, 2004

(54) Vigilant vector system

(76) Inventors: Michael Ian Phillips, Tampa, FL (US); Yi Tang, Gainesville, FL (US)

Correspondence Address: Akerman Senterfitt, Stanley A. Kim, Ph.D., Esq., Suite 400, 222 Lakeview Avenue, West Palm Beach, FL, 33402-3188, US

(21) Appl. No.: 10/659,621

(22) Filed: Sep. 10, 2003

Related U.S. Application Data
(60) Provisional application No. 60/409,688, filed on Sep. 11, 2002.

(51) Int. Cl. C12N 15/861

(52) U.S. Cl. 435/456

(57) ABSTRACT

A system for expressing a heterologous gene in a cell type-specific and inducible manner features at least one vector that includes a nucleic acid encoding a switch/biosensor, a nucleic acid encoding a tissue-specific promoter, a nucleic acid encoding the heterologous gene, and a nucleic acid encoding a gene amplification component. The switch/biosensor allows the system to regulate expression of the heterologous gene in response to a stimulus. The tissue-specific promoter allows the system to selectively express the heterologous gene in a particular cell or tissue type. And the gene amplification component induces expression of the heterologous gene at a level sufficient to exert a physiological effect on a tissue administered the system.

### CROSS-REFERENCE TO RELATED APPLICATIONS

The present application claims the priority of U.S. provisional patent application No. 60/409,688 entitled “Vigilant Viral Vector System,” and filed on Sep. 10, 2002.

### STATEMENT AS TO FEDERALLY SPONSORED RESEARCH

The invention was made with U.S. government support under grant number R37 HL27334 awarded by the National Institutes of Health. The U.S. government may have certain rights in the invention.

### FIELD OF THE INVENTION

The invention relates to the fields of molecular biology, gene therapy, and virology. More particularly, the invention relates to compositions and methods for modulating cellular physiology using a stimulus-responsive viral vector system to deliver and amplify a heterologous gene in a host cell.

### BACKGROUND OF THE INVENTION

In conventional gene therapy strategies, expression of a heterologous gene (i.e., a transgene) is usually constitutive and non-specific with regard to particular cell types. The resulting non-physiological protein expression pattern can cause problems such as inappropriate down-regulation of effector systems, cellular toxicity, and pathophysiology (Isgaard et al., Endocrinology 123:2605-2610, 1988; Wolf et al., Mech Ageing Dev. 68:71-87, 1993). Gene therapy systems that regulate transgene expression in an inducible and tissue-specific manner have therefore been sought.

Previously proposed inducible gene expression systems usually utilize two or more co-expressed vectors wherein one vector expresses a chimeric transcription factor which functions as a gene switch (regulated by the presence or absence of a small molecule) and another vector contains a reporter gene operably linked to a promoter that is responsive to the gene switch. An example of an inducible gene expression system is the tet on-off system, a tetracycline (tet)-regulated system, which features either a tet-repressible or a tet-activating mechanism. In the presence of antibiotic (i.e., tetracycline), a tet-repressor containing transactivator (tTA) does not bind to a tTA-dependent promoter and transcription is blocked. By withdrawing tetracycline, transcription is activated (Gossen M. and Bujard H. Proc Natl Acad Sci USA. 89:5547-5551, 1992). In the tet-activating system, the transactivator contains a mutant tet repressor that requires doxycycline, a tet derivative, for specific DNA binding. Transgene expression is turned on in the presence of doxycycline and turned off in the absence of doxycycline (Gossen et al., Science. 268:1766-1769, 1995).

Other known inducible gene expression systems include: 1) the ecdyson-inducible system (No et al., Proc Natl Acad Sci USA 93:3346-3351, 1996) which involves a retinoid X receptor and a synthetic analog of ecdysone; 2) the antiprogestin-regulated gene switch, which involves a progesterone receptor ligand-binding domain fusion protein and progesterone antagonists (Vegeto et al., Cell. 69:703-713, 1992) such as RU486/mifeprostone (Wang et al., Nat Biotechnol. 15:239-243, 1997); and 3) the dimerization-based gene switch system that involves FKBP-DBD and FRAP fusion proteins (Rivera et al., Nat. Med. 2:1028-1032, 1996) that are associated by rapamycin, resulting in a heterodimeric protein complex that activates transcription from an inducible promoter.

The weakness of all of these systems is that they require exogenous drugs as inducers. The use of exogenous drugs results in unwanted side effects, thereby defeating the purpose of gene therapy which is to deliver highly specific and non-toxic gene expression. For example, tetracycline deposits in bone and stains teeth (Cohlan SQ. Teratology. 15:127-129, 1977). Long term administration may lead to bacterial resistance. RU486/mifeprostone is a progesterone antagonist (Vegeto et al., Cell 69:703-713, 1992). And rapamycin exhibits growth inhibitory and immunosuppressive effects.

Tissue-specific gene expression can be achieved using known tissue-specific promoters. A major problem with current tissue-specific promoters is that they induce relatively weak transgene expression compared to non-tissue-specific promoters such as the cytomegalovirus (CMV) promoter. In many cases, the low level of transgene expression is insufficient for achieving a therapeutic effect. An ideal promoter would therefore be one that is inducible by a physiological signal and achieves high transgene expression levels in a tissue-specific manner.

### SUMMARY

The invention relates to a “Vigilant Vector” system for delivering therapeutic genes to target tissues in an inducible and tissue-specific manner. This system employs one or more vectors that contain a gene switch/biosensor, a tissue-specific promoter, and a gene amplification system. The gene switch/biosensor allows transgene expression to be regulated by a stimulus (e.g., a physiological stimulus). Thus, for example, transgene expression can be increased or decreased in response to a particular event (e.g., a physiological signal such as an increase in glucose concentration or a decrease in oxygen concentration). The tissue-specific promoter allows the transgene to be preferentially expressed in a given target tissue, thereby avoiding expression in other tissues that might be harmful to the subject. The gene amplification system allows production of a sufficient amount of the transgene to cause a desired (e.g., therapeutic) result. The biosensor/switch component allows for activation of multiple genes simultaneously.

While the system of the invention may be used in a myriad of different applications (as treatment or prophylaxis for diseases such as diabetes, cancer, stroke, pulmonary fibrosis, arthritis, atherosclerosis and inflammation), one application utilizes a dual recombinant Adeno-Associated Virus (rAAV) vector system to detect and respond to hypoxia in cardiac tissue. The first rAAV vector in this system is the “sensor” vector. This vector contains a cardiac-specific promoter linked to a sequence encoding an oxygen-sensitive chimeric transactivator, which is termed a redox sensor toggle (RST). “Redox” refers to reduced oxygen availability and “toggle” refers to the ability of the system to switch on genes. The RST contains a GAL4 DNA-binding domain (DBD), an oxygen-dependent degradation domain (ODD), and a p65 activation domain (p65 AD). The second rAAV vector of the system is the “effector” vector which contains a cardioprotective gene linked to a GAL4 upstream activating sequence (UAS). The sensor rAAV vector expresses the chimeric transactivator specifically in the heart. In response to hypoxia, the transactivator binds to the GAL4 UAS sequence in the effector rAAV vector, resulting in the expression of the cardioprotective gene.

Accordingly, the invention features a system for expressing a heterologous gene in a cell type-specific and inducible manner. The system has at least one vector that includes a nucleic acid encoding a switch/biosensor, a nucleic acid encoding a tissue-specific promoter, a nucleic acid encoding the heterologous gene, and a nucleic acid encoding a gene amplification component. The switch/biosensor allows the system to regulate expression of the heterologous gene in response to a stimulus. The tissue-specific promoter allows the system to selectively express the heterologous gene in a particular cell or tissue type. And the gene amplification component induces expression of the heterologous gene at a level sufficient to exert a physiological effect on a tissue administered the system.

In the system, the at least one vector can include one or more AAV vectors, e.g., a first AAV vector and a second AAV vector. In the latter arrangement, the first AAV vector can include the nucleic acid encoding the switch/biosensor and/or the nucleic acid encoding the tissue-specific promoter. In a further variation, the switch/biosensor, the tissue-specific promoter, and transactivator are encoded by nucleic acids on the first AAV vector, and the regulatory element and the heterologous gene are encoded by nucleic acids on the second AAV vector.

In the invention, the switch/biosensor can include an ODD, the tissue-specific promoter can include a cardiac-specific promoter (e.g., MLC-2v), and the gene amplification component can include a transactivator and a regulatory element responsive to the transactivator. The regulatory element can be operatively linked to the heterologous gene such that binding of the transactivator to the regulatory element increases expression of the heterologous gene. The transactivator can include a GAL4 DBD and/or a p65 AD, while the regulatory element can include a GAL4 UAS and/or an adenovirus (Ad) E1b TATA element.

The system of the invention can be located in at least one cell such as one in an animal subject. In another aspect, the invention features a method for expressing a heterologous gene in a cell in a cell type-specific and inducible manner. The method includes the step of administering to the cell a system of the invention.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of virology terms can be found in Granoff and Webster, Encyclopedia of Virology, 2nd edition, Academic Press: San Diego, Calif., 1999; and Tidona and Darai, The Springer Index of Viruses, 1st edition, Springer-Verlag: New York, 2002. Commonly understood definitions of microbiology can be found in Singleton and Sainsbury, Dictionary of Microbiology and Molecular Biology, 3rd edition, John Wiley & Sons: New York, 2002.

As used herein, the phrase “nucleic acid” means a chain of two or more nucleotides such as RNA (ribonucleic acid) and DNA (deoxyribonucleic acid). As used herein, the term “gene” means a nucleic acid molecule that codes for a particular protein, or in certain cases, a functional or structural RNA molecule. By the phrase “heterologous gene” or “heterologous nucleic acid” is meant a gene or nucleic acid that is to be introduced into and expressed in a host cell, e.g., in a gene therapy protocol.

As used herein, “protein” or “polypeptide” mean any peptide-linked chain of amino acids, regardless of length or post-translational modification, e.g., glycosylation or phosphorylation.

By the term “promoter” is meant a DNA regulatory sequence to which RNA polymerase binds, initiating transcription of a downstream (3′ direction) coding sequence. A “tissue-specific promoter” is one that exerts a regulatory effect on transcription in only one type of tissue or a small subset of tissues.

A first nucleic acid sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

As used herein, the term “vector” refers to a molecule capable of transporting a nucleic acid to which it has been linked, e.g., a plasmid. An “rAAV vector” is a vector that includes nucleic acid sequences derived from AAV.

By use of “virion” is meant a completed virus particle that contains a nucleic acid and a protein coat (capsid). An “rAAV virion” is a virion that includes nucleic acid sequences and/or proteins derived from AAV.

As used herein, the terms “inverted terminal repeat,” “terminal repeat” or “TR” mean a nucleic acid sequence derived from an AAV that is required in cis for replication and packaging of AAV.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is two schematic models of Vigilant Vector systems. A. Single vector model contains a hypoxia switch, cardiac promoter, and cardioprotective transgene adapted between the TRs of AAV. B. Double vector model. In the sensor AAV (top vector), the cardiac promoter controls the expression of an oxygen-sensitive transactivator. The transactivator is a fusion protein, which binds to a transcription sequence in the effector AAV (bottom vector) and activates the transcription of a cardioprotective transgene.

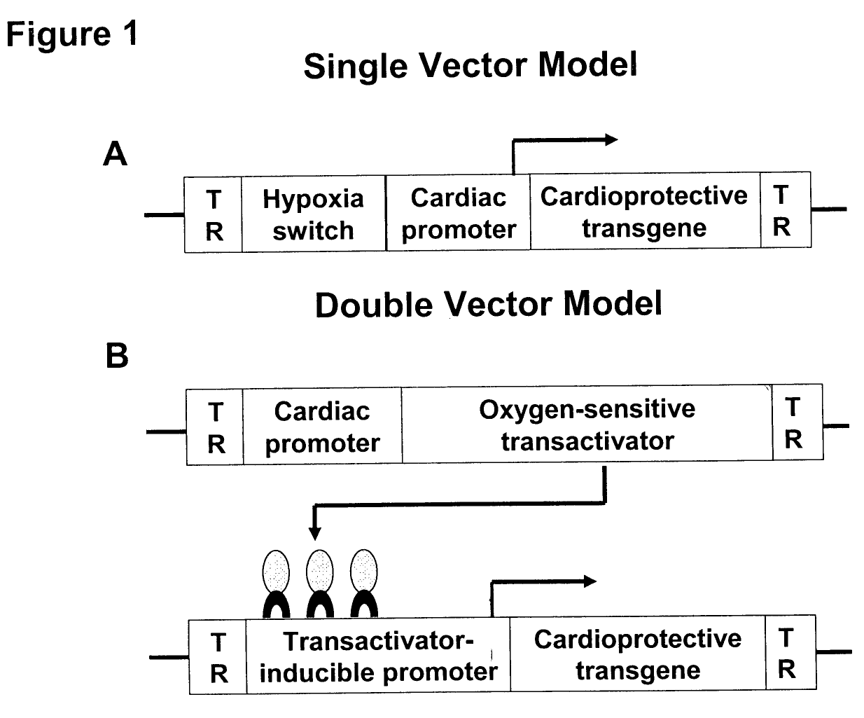

FIG. 2 is a schematic illustration of the hypoxia switch termed the MLC RST system. The system includes a transactivator plasmid and a reporter plasmid. In the transactivator plasmid, the MLC-2v promoter controls the expression of an oxygen sensitive transcriptional factor (i.e., RST). The RST is composed of a GAL4 DBD, ODD and p65 AD. It degrades under normoxia, but accumulates under hypoxia and activates the inducible promoter containing GAL4 UAS in the reporter plasmid. The RST can be switched on by hypoxia and off by normoxia and when it is switched on, it amplifies transgene expression.

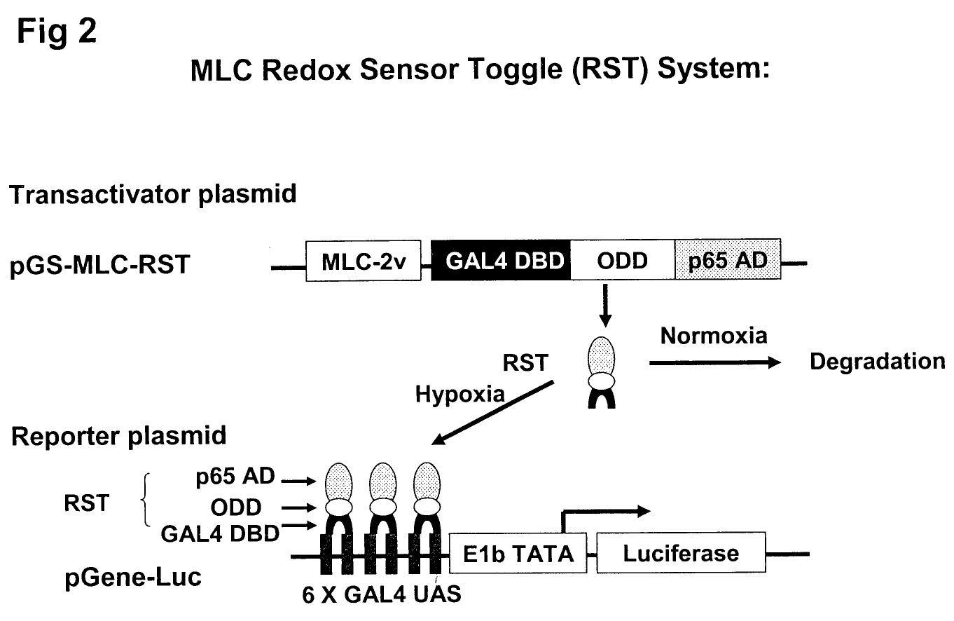

FIG. 3 is a graph showing that the RST up-regulates reporter gene expression in response to hypoxia. Relative luciferase activity represents the ratio of firefly luciferase activity versus Renilla luciferase activity. Induction ratio by hypoxia was determined by comparing the relative luciferase activity in cells at 1% O₂ to 20% O₂. (mean±S.D.; n=three samples)

FIG. 4 is a graph showing the hypoxia-inducible function of the RST in adult rat cardiomyocytes. Relative luciferase activity represents the ratio of firefly luciferase activity versus Renilla luciferase activity. Induction ratio by hypoxia was determined by comparing the relative luciferase activity in cells at 1% O₂ to 20% O₂. (mean±S.D.; n=three independent experiments).

FIG. 5 is a graph showing the responsiveness of the RST to hypoxia in human cells. Relative luciferase activity represents the ratio of firefly luciferase activity versus Renilla luciferase activity. Expression at 1% relative to 20% O₂ was calculated (Induction).

FIG. 6 is a graph showing the time course of increased luciferase expression by the RST under hypoxia. The increased luciferase activity in the hypoxia-treated group is expressed as increase percent over that in the control cells. (mean±S.D.; n=6-9 samples) The data is concluded from [time of gene expression]−[time for the media to reach 1% O₂] after the O₂ level was reduced.

FIG. 7 is a graph showing the switch on-and-off function of the RST. The increased luciferase expression in the treated group is expressed as increase percent over control. (mean±S.D.; n=6-9 samples)

FIG. 8 is a graph showing the cardiac-specific expression that can be achieved with the MLC RST. (mean±S.D., n=3 samples)

FIG. 9 is a graph showing that the RST amplifies the power of the MLC-2v promoter. X-Fold represents the ratio of relative luciferase activity in MLC-RST versus in MLC-Luc. (mean±S.D., n=3 samples)

FIG. 10 is a graph showing that a single RST transactivator can switch on multiple reporter genes in response to hypoxia. The expression under hypoxia was expressed as X-fold compared to normoxia. Induction represents the ratio of the expression of reporter gene under hypoxia versus normoxia. (mean±S.D., n=3 samples)

### DETAILED DESCRIPTION

The invention provides a vector-based system for expressing a heterologous gene in a target tissue or cell in an inducible and tissue-specific manner. This system employs one or more vectors that collectively include a switch/biosensor that allows the system to regulate transgene expression in response to a signal, a tissue-specific promoter for selectively expressing the transgene in a particular cell or tissue type, and a gene amplification system that increase transgene expression to a level sufficient to exert a physiological (e.g., therapeutic) effect. The invention is illustrated herein with a two rAAV vector system that detects low oxygen levels in cardiac tissue as might occur in myocardial infarction and responds by turning on expression of a gene encoding a cardio-protective protein. The first vector contains a cardiac tissue-specific promoter operably linked to a sequence encoding a transactivator that includes a GAL4-DBD, an ODD, and a p65 AD; and the second vector contains a GAL4 UAS operably linked to the cardioprotective gene. When cardiac cells harboring the system become hypoxic, the transactivator binds to the UAS sequence and thereby causes expression of the cardioprotective gene.

The below described preferred embodiments illustrate various adaptations of this system using the two rAAV vector cardio-protective system as an example. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy Methods: ed. M. I. Phillips, Vol. 436, Methods in Enzymology, Academic Press, 2002; Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Vectors

The featured system for expressing a heterologous gene in a target tissue or cell in an inducible and tissue-specific manner utilizes one, two, three or more vectors that collectively encode a switch/biosensor, a tissue-specific promoter, and a gene amplification system. The vector can take the form of any vehicle that can be used to introduce and express a heterologous gene in a host cell. Several are known in the art including viral vectors and non-viral vectors. More than one promoter can be present in a vector, and hence more than one heterologous gene can be expressed by a vector. Further, the vector can include a sequence which encodes a signal peptide or other moiety which facilitates the secretion of the heterologous gene product from the host cell.

Viral vectors that exhibit low toxicity to the host cell and are capable of producing high levels of a transgene are preferred as such vectors have been extensively studied and characterized. Examples of viral vector methods and protocols are reviewed in Kay et al. Nature Medicine 7:33-40, 2001.

AAV vectors are presently preferred viral vectors for use in the system of the invention because they have proven to be safe, stable and capable of providing long-lasting transgene expression in other applications. Moreover AAV vectors are able to efficiently transduce a number of different target cells and can integrate into a host cell's genome in a site-specific manner. Methods for use of rAAV vectors are discussed, for example, in Tal, J., J. Biomed. Sci. 7:279-291, 2000 and Monahan and Samulski, Gene Therapy 7:24-30, 2000. For use in a two vector system, a first preferred rAAV vector (the sensor vector) contains a pair of AAV TRs flanking at least one cassette containing a promoter (e.g. MLC-2v) which directs tissue (e.g., heart) or cell-specific (e.g., cardiac myocyte) expression operably linked to a nucleotide sequence encoding a chimeric transactivator (e.g., GAL4 DBD/ODD/p65AD). The second preferred rAAV vector (effector vector) of the system contains a pair of AAV TRs flanking several copies of a GAL4 UAS linked to a TATA element and a heterologous gene (e.g., a cardioprotective or cytoprotective gene). After administration to a cell or tissue, the DNA of the rAAV vectors, including the TRs, the promoter and heterologous gene become integrated into the host genome (Wu et al, J. Virology 72(7):5919-5926, 1998).

The rAAV vectors used in the invention are nucleic acid sequences that include those AAV sequences required in cis for replication and packaging (e.g., functional TRs) of the DNA into a virion. Useful rAAV vectors have one or more of the AAV WT genes deleted in whole or in part, but retain functional flanking TR sequences. The AAV nucleic acids, proteins, vectors, and virions used in the invention may be derived from any of several AAV serotypes including 1, 2, 3, 4, 5, 6, 7, and 8. Genes from these serotypes can be isolated using standard methods (Chiorini et al., J Virol. 73:1309-19, 1999; Rutledge et al., J Virol. 72:309-19, 1998; Xiao et al., J Virol. 73:3994-4003, 1999; Muramatsu et al., Virology 221:208, 1996; and Chiorini, et al., J. Virol. 71:6823, 1997; Sambrook and Russell supra). Vectors containing rep genes and TRs derived from serotype 2 are particularly preferred because serotype 2 vectors have been characterized in great detail and constructs derived from serotype 2 are commonly available. Particular AAV vectors and AAV proteins of different serotypes are discussed in Chao et al., Mol. Ther. 2:619-623, 2000; Davidson et al., Proc. Nat'l Acad. Sci. USA 97:3428-3432, 2000; and Xiao et al., J. Virol. 72:2224-2232, 1998.

Also useful in the system of the invention are rAAV virions that have mutations within the virion capsid. For example, suitable rAAV mutants may have ligand insertion mutations for the facilitation of targeting rAAV virions to specific cell types (e.g., cardiac myocytes). Pseudotyped rAAV virions that have mutations within the capsid may also be used in compositions and methods of the invention. Pseudotyped rAAV virions contain an rAAV vector derived from a particular serotype that is encapsidated within a capsid containing proteins of another serotype. Methods of making AAV capsid mutants are known, and include site-directed mutagenesis (Wu et al., J. Virol. 72:5919-5926); molecular breeding, nucleic acid, exon, and DNA family shuffling (Soong et al., Nat. Genet. 25:436-439, 2000; Coco et al., Nature Biotech. 2001; 19:354; and U.S. Pat. Nos. 5,837,458; 5,811,238; and 6,180,406; Kolkman and Stemmer, Nat. Biotech. 19:423-428, 2001; Fisch et al., Proc. Nat'l Acad. Sci. USA 93:7761-7766, 1996; Christians et al., Nat. Biotech. 17:259-264, 1999); ligand insertions (Girod et al. Nat. Med. 9:1052-1056, 1999); cassette mutagenesis (Rueda et al. Virology 263:89-99, 1999; Boyer et al., J. Virol. 66:1031-1039, 1992); and the insertion of short random oligonucleotide sequences.

### Additional Vectors

In addition to AAV, Ad vectors might also be used in the system of the invention. Methods for using recombinant Ad as gene therapy vectors are discussed, for example, in W. C. Russell, Journal of General Virology 81:2573-2604, 2000, and Bramson et al., Curr. Opin. Biotechnol. 6:590-595, 1995. Ad vectors have been shown to be capable of highly efficient gene expression in target cells and allow for a large coding capacity of heterologous DNA. An especially useful form of recombinant Ad is a “gutless”, “high-capacity”, or “helper-dependent” Ad vector which has all viral coding sequences deleted, and contains the viral inverted terminal repeats (ITRs), therapeutic gene (e.g., cardioprotective gene) sequences (up to 28-32 kb) and the viral DNA packaging sequence. Variants of such recombinant Ad vectors such as vectors containing tissue-specific (e.g., heart) enhancers and promoters operably linked to nucleotide sequences encoding a chimeric transactivator (e.g., GAL4 DBD/ODD/p65AD) are also within the invention. Another preferred Ad vector contains a viral packaging sequence and a pair of Ad ITRs which flank several copies of a GAL4 UAS linked to a TATA element and therapeutic gene (e.g., cardioprotective gene). Also useful is an Ad vector that contains both: 1) a tissue-specific (e.g., heart) promoter operably linked to a nucleotide sequence encoding a chimeric transactivator (e.g., GAL4 DBD/ODD/p65AD) and 2) several copies of a GAL4 UAS linked to a TATA element and therapeutic gene (e.g., cardioprotective gene).

Herpes Simplex Virus (HSV) vectors might also be used in the invention. Methods for use of HSV vectors are discussed, for example, in Cotter and Robertson, Curr. Opin. Mol. Ther. 1:633-644, 1999. HSV vectors deleted of one or more IE genes are non-cytotoxic, persist in a state similar to latency in the host cell, and afford efficient host cell transduction. Recombinant HSV vectors allow for approximately 30 kb of coding capacity. A first preferred HSV vector is engineered from HSV type I, is deleted of the IE genes and contains a tissue-specific (e.g., heart) promoter operably linked to a nucleotide sequence encoding a chimeric transactivator (e.g., GAL4 DBD/ODD/p65AD). A second preferred HSV vector is engineered from HSV type I, is deleted of the immediate early genes (IE) and contains several copies of a GAL4 UAS linked to a TATA element and therapeutic gene (e.g., cardioprotective gene). Another example of a useful HSV vector is one that contains both: 1) a tissue-specific (e.g., heart) promoter operably linked to a nucleotide sequence encoding a chimeric transactivator (e.g., GAL4 DBD/ODD/p65AD) and (2) several copies of a GAL4 UAS linked to a TATA element and therapeutic gene (e.g., cardioprotective gene). HSV amplicon vectors may also be used according to the invention. Typically, HSV amplicon vectors are approximately 15 kb in length, possess a viral origin of replication and packaging sequences.

The invention also provides for use of retroviral vectors, including Murine Leukemia Virus-based vectors. Methods for use of retrovirus-based vectors are discussed, for example, in Hu and Pathak, Pharmacol. Rev. 52:493-511, 2000 and Fong et al., Crit. Rev. Ther. Drug Carrier Syst. 17:1-60, 2000. Retroviral vectors according to the invention may contain up to 8 kb of heterologous (e.g., therapeutic) DNA, in place of the viral genes. Heterologous may be defined in this context as any nucleotide sequence or gene which is not native to the retrovirus. The heterologous DNA may include a tissue-specific promoter (e.g., cardiac-specific) operably linked to a nucleotide sequence encoding a chimeric transactivator (e.g., GAL4 DBD/ODD/p65AD) and may encode a ligand to a cell-specific receptor. In the same or a separate retrovirus vector, the heterologous DNA may include several copies of a GAL4 UAS linked to a TATA element and therapeutic gene (e.g., cardioprotective gene). Another example of a useful retrovirus vector is one that contains both: 1) a tissue-specific (e.g., heart) promoter operably linked to a nucleotide sequence encoding a chimeric transactivator (e.g., GAL4 DBD/ODD/p65AD) and (2) several copies of a GAL4 UAS linked to a TATA element and therapeutic gene (e.g., cardioprotective gene). The retroviral particle may be pseudotyped, and may contain a viral envelope glycoprotein from another virus, in place of the native retroviral glycoprotein. A retroviral vector useful in the invention may integrate into the genome of the host cell.

Viral vectors utilized in the present invention may also include replication-defective lentiviral vectors, including HIV. Methods for use of lentiviral vectors are discussed, for example, in Vigna and Naldini, J. Gene Med. 5:308-316, 2000 and Miyoshi et al., J. Virol. 72:8150-8157, 1998. Lentiviral vectors are capable of infecting both dividing and non-dividing cells and efficient transduction of epithelial tissues of humans. HIV vectors have been shown to infect cardiac cells. Lentiviral vectors according to the invention may be derived from human and non-human (including SIV) lentiviruses. A preferred lentiviral vector of the present invention may include nucleic acid sequences required for vector propagation in addition to a tissue-specific promoter (e.g., heart) operably linked to a nucleotide sequence encoding a chimeric transactivator (e.g., GAL4 DBD/ODD/p65AD). A second preferred lentiviral vector may include nucleic acid sequences required for vector propagation in addition to several copies of a GAL4 UAS linked to a TATA element and therapeutic gene (e.g., cardioprotective gene). Another example of a useful lentiviral vector is one that contains both: 1) a tissue-specific (e.g., heart) promoter operably linked to a nucleotide sequence encoding a chimeric transactivator (e.g., GAL4 DBD/ODD/p65AD) and (2) several copies of a GAL4 UAS linked to a TATA element and therapeutic gene (e.g., cardioprotective gene). These sequences may include the viral LTRs, primer binding site, polypurine tract, att sites and encapsidation site. The lentiviral vector may be packaged into any suitable lentiviral capsid. The vector capsid may contain viral envelope proteins from other viruses, including Murine Leukemia Virus (MLV) or Vesicular Stomatitis Virus (VSV). The use of the VSV G-protein yields a high vector titer and results in greater stability of the vector virus particles.

Other viral vectors that might be used in the invention are Alphaviruses, including Semliki Forest Virus (SFV) and Sindbis Virus (SIN). Methods for use of Alphaviruses are described, for example, in Lundstrom, K., Intervirology 43:247-257, 2000 and Perri et al., Journal of Virology 74:9802-9807, 2000. Alphavirus vectors typically are constructed in a format known as a replicon. Such replicons may contain Alphavirus genetic elements required for RNA replication, as well as tissue-specific (e.g., cardiac) therapeutic gene expression. Within the Alphavirus replicon, a tissue-specific promoter (e.g., cardiac-specific) may be operably linked to a nucleotide sequence encoding a chimeric transactivator (e.g., GAL4 DBD/ODD/p65AD) and may encode a ligand to a cell-specific receptor. In the same or a separate Alphavirus vector, the heterologous DNA may include several copies of a GAL4 UAS linked to a TATA element and therapeutic gene (e.g., cardioprotective gene). Another example of a useful Alphavirus vector is one that contains both: 1) a tissue-specific (e.g., heart) promoter operably linked to a nucleotide sequence encoding a chimeric transactivator (e.g., GAL4 DBD/ODD/p65AD) and (2) several copies of a GAL4 UAS linked to a TATA element and therapeutic gene (e.g., cardioprotective gene). Recombinant, replication-defective Alphavirus vectors are capable of high-level heterologous (e.g., therapeutic) gene expression, and can infect a wide host cell range. Alphavirus replicons according to the invention may be targeted to specific cell types (e.g., cardiac myocytes) by displaying on their virion surface a functional heterologous ligand or binding domain that would allow selective binding to target cells expressing the cognate binding partner. Alphavirus replicons according to the invention may establish latency, and therefore long-term tissue-specific therapeutic gene expression in the host cell. The replicons may also exhibit transient tissue-specific therapeutic gene expression in the host cell. A preferred Alphavirus vector or replicon of the invention is on that is noncytopathic.

To combine advantageous properties of two viral vector systems, hybrid viral vectors may be used to deliver stimulus-responsive vector sequences to a target tissue (e.g., heart). Standard techniques for the construction of hybrid vectors are well-known to those skilled in the art. Such techniques can be found, for example, in Sambrook and Russell, supra, or any number of laboratory manuals that discuss recombinant DNA technology. Double-stranded AAV genomes in Ad capsids containing a combination of AAV and Ad ITRs may be used to transduce cells. In another variation, an AAV vector may be placed into a “gutless”, “helper-dependent” or “high-capacity” Ad vector. Ad/AAV hybrid vectors are discussed in Lieber et al., J. Virol. 73:9314-9324, 1999. Retroviral/Ad hybrid vectors are discussed in Zheng et al., Nature Biotechnol. 18:176-186, 2000. Retroviral genomes contained within an Ad may integrate within the host cell genome and effect stable, tissue-specific therapeutic gene expression.

Several non-viral methods for introducing nucleic acids into host cells might also be used in the invention. For a review of non-viral methods, see Nishikawa and Huang, Human Gene Ther. 12:861-870, 2001 and M. I. Phillips, Gene Therapy Methods, Academic press, 2002. For example, various techniques employing plasmid DNA for the introduction of transactivator (e.g., GAL4 DBD/ODD/p65AD) and reporter sequences (e.g., several copies of a GAL4 UAS linked to a TATA element and therapeutic gene (e.g., cardioprotective gene) into cells are provided for in the invention. Such techniques are generally known in the art and are described in references such as Ilan, Y., Curr. Opin. Mol. Ther. 1:116-120, 1999, Wolff, J. A., Neuromuscular Disord. 7:314-318, 1997 and Arztl, Z., Fortbild Qualitatssich 92:681-683, 1998.

Methods involving physical techniques for the introduction of a vector system into a host cell can be adapted for use in the present invention. The particle bombardment method of gene transfer involves an Accell device (i.e., gene gun) to accelerate DNA-coated microscopic gold particles into target tissue, including the heart. Particle bombardment methods are described in Yang et al., Mol. Med. Today 2:476-481 1996 and Davidson et al., Rev. Wound Repair Regen. 6:452-459, 2000. Cell electropermeabilization (also termed cell electroporation) may be employed for transactivator (e.g., GAL4 DBD/ODD/p65AD) and reporter sequence (e.g., several copies of a GAL4 UAS linked to a TATA element and therapeutic gene (e.g., cardioprotective gene) delivery into cells of tissues. This technique is discussed in Preat, V., Ann. Pharm. Fr. 59:239-244 2001 and involves the application of pulsed electric fields to cells to enhance cell permeability, resulting in exogenous polynucleotide transit across the cytoplasmic membrane.

Synthetic gene transfer molecules according to the invention can be designed to form multimolecular aggregates with plasmid DNA (e.g., nucleotides encoding a GAL4 DBD/ODD/p65AD transactivator and several copies of a GAL4 UAS linked to a TATA element and therapeutic gene) and to bind the resulting particles to the target cell (e.g., cardiac myocyte) surface in such a way as to trigger endocytosis and endosomal membrane disruption. Polymeric DNA-binding cations (including polylysine, protamine, and cationized albumin) can be linked to cardiac-specific targeting ligands and trigger receptor-mediated endocytosis into cardiac myocytes. Methods involving polymeric DNA-binding cations are reviewed in Guy et al., Mol. Biotechnol. 3:237-248, 1995 and Garnett, M. C., Crit. Rev. Ther. Drug Carrier Syst. 16:147-207, 1999. Cationic amphiphiles, including lipopolyamines and cationic lipids, may provide receptor-independent transfer of transactivator (e.g., GAL4 DBD/ODD/p65AD) and reporter sequences (e.g., several copies of a GAL4 UAS linked to a TATA element and therapeutic gene (e.g., cardioprotective gene) into target cells (e.g., cardiac myocytes). Preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. Suitable methods can also include use of cationic liposomes as agents for introducing DNA or protein into cells. For therapeutic gene delivery, DNA may also be coupled to an amphipathic cationic peptide (Fominaya et al., J. Gene Med. 2:455-464, 2000).

Methods that involve both viral and non-viral based components may be used according to the invention. An Epstein Barr Virus (EBV)-based plasmid for therapeutic gene delivery is described in Cui et al., Gene Therapy 8:1508-1513, 2001. A method involving a DNA/ligand/polycationic adjunct coupled to an Ad is described in Curiel, D. T., Nat. Immun. 13:141-164, 1994.

Other techniques according to the invention may be based on the use of tissue (e.g., heart)-specific ligands. Synthetic peptides or polypeptides may be used as ligands in targeted delivery of DNA and proteins to heart-specific receptors. Complexes of protein and ligand or plasmid DNA and ligand can mediate protein and DNA transfer into cardiac cells. For example, cardiac-Adrenergic receptors bind isoproterenol and may be used to target delivery to cardiac cells (Kim et al., J. Biol. Chem. June 4, epub ahead of print, 2002; and Eckhart et al., Mol. Pharmacol. 61:749-758, 2002).

Methods involving ultrasound contrast agent delivery vehicles may be used in the invention. Such methods are discussed in Newman et al., Echocardiography 18:339-347, 2001 and Lewin et al. Invest. Radiol. 36:9-14, 2001. Gene-bearing microbubbles, when exposed to ultrasound, cavitate and locally release a therapeutic agent. Attachment of a heart cell-targeting moiety to the contrast agent vehicle may result in site-specific (e.g., heart) therapeutic gene expression.

Delivery of a vector system, according to the invention, can also involve methods of ex vivo gene transfer using stem cells and progenitor cells. Such methods involve the isolation and expansion of selected stem or progenitor cells, introduction of a therapeutic gene(s) into the cells ex vivo, and return of the genetically modified cells to the host. Stem cells including hematopoietic stem cells and mesenchymal stem cells have been shown to differentiate into functional cardiomyocytes when in the presence of differentiated cardiomyocytes. See Orlic et al., Nature 410:701-705,2001; and Condorelli et al., Proc Nat'l Acad. Sci. USA98:10733-10738, 2001. Mesenchymal stem cells (MSC) are particularly suited as a delivery vehicle for gene transfer. MSC can be obtained from a bone marrow aspirate taken from the iliac crest of adult humans and are well tolerated when transplanted to humans and animals. MSC can be ex vivo expanded to large numbers and retain the ability to differentiate into cardiac, bone, adipocytes and muscle cells in vitro and in vivo. Mosca et al., Clin. Orthop. 379 Suppl:S71-90, 2000; and Le Blanc, K., Lakartidningen 99:1318-1324, 2002. Muscle-derived stem cells (MDSCs) are also capable of delivering therapeutic genes and differentiating into a cardiomyocyte lineage within the heart. MDSCs are easily accessible via simple biopsy of the patient's own muscle. The isolation and characteristics of MDSCs and their use in regenerative medicine are reviewed in Sakai et al., Trends Cardiovasc. Med. 12:115-120, 2002. Based on their differentiation properties and facility of ex vivo expansion, human bone marrow mesenchymal progenitor cells (MPC) are also a suitable vehicle for delivering therapeutic genes to the bone marrow and other mesenchymal tissues. For example, MPC can be expanded ex vivo and infected with an rAAV virion of the invention or transduced with a stimulus-responsive rAAV vector(s). Conget et al., Exp. Hematol. 28:382-390, 2000.

Colonies of cells derived from vector-transduced cells can be assessed by PCR, flow cytometry and immunochemistry. The functional capacity of vector-transduced cells can be examined by quantifying clonogeneic efficiency and proliferative capacity. Frey et al., Blood 91:2781-2792, 1998. Following implantation of the transduced cells into the host, therapeutic gene expression can be analyzed by immunocytochemistry and RT-PCR of tissue samples. Condorelli et al., Proc Nat'l Acad. Sci. USA 98:10733-10738, 2001. PCR techniques may be used to identify and locate implanted cells that carry a particular vector and/or heterologous gene. See Hou et al., Proc Nat'l Acad. Sci. USA 96:7294-7299, 1999.

### Gene Switch/Biosensor

The vector system of the invention includes a gene switch/biosensor to sense the presence of a stimulus (e.g., low oxygen) and relay a message to another component of the system to start or increase expression of a heterologous gene. In the hypoxia-sensing, cardio-protective double AAV vector system described herein, the gene switch/biosensor forms part of the oxygen-sensitive chimeric transactivator fusion protein that includes a GAL4 DBD, ODD, and p65 AD. The ODD portion of this molecule controls ubiquitin-proteosome-mediated degradation of HIF-1α and confers oxygen-dependent instability when fused to a stable protein (GAL4 in this case). The chimeric transactivator encoded in a first vector that is introduced into a target cell (cardiomyocyte) or tissue (heart) is constitutively produced in that cell or tissue (in a tissue-specific or selective manner if a tissue-specific promoter is used). A second vector encoding a heterologous gene operably linked to an element responsive to the chimeric transactivator (a GAL4 UAS sequence) is also introduced into the target cell or tissue. In the presence of a stimulus (in this case hypoxia), the transactivator (GAL4-DBD/ODD/p65 AD) binds to the element responsive to the chimeric transactivator (GAL4 UAS) and induces expression of the heterologous gene. In the absence of the stimulus (i.e., under normoxic conditions), ODD destabilizes the chimeric transactivator and thereby prevents it from inducing expression of the heterologous gene.

Based on the teachings herein, a number of other gene switch/biosensors can be envisaged. For example, switch responsive to high or low levels of glucose or those responsive to one or more inflammatory cytokines can be constructed, e.g., by replacing the ODD portion of the above-described sensor with another element that is sensitive to another stimulus.

### Tissue-Specific Regulatory Elements

The invention utilizes tissue-specific regulatory elements to direct tissue-specific expression of other elements of the system, e.g., the gene switch/sensor or the heterologous gene. To illustrate, to express genes specifically in the heart, a number of cardiac-specific regulatory elements can be used. An example of a cardiac-specific promoter is the ventricular form of MLC-2v promoter. The MLC-2v promoter is 3.0 kb and contains a TATA box and several conserved cis regulatory sequences including HF-1. Zhu et al., Mol. Cell Biol. 13:4432-4444, 1993; Navankasattusas et al., Mol. Cell Biol. 12:1469-1479, 1992. The proximal 250 bp containing the TATA box and regulatory sequences are sufficient to confer cardiac muscle-specificity as well as ventricular-restricted specificity. Lee et al., J. Biol. Chem. 267:15875-15885, 1992; and Franz et al., Cardiovasc. Res. 35:560-566, 1997. Other MLC-2v promoter sequences within the invention are variants of MLC-2v such as those that are fragments, analogs and derivatives of native MLC-2v. Such variants may be, e.g., a naturally occurring allelic variant of native MLC-2v, a homolog of native MLC-2v or a non-naturally occurring variant of native MLC-2v. For example, a 281 bp fragment of the native MLC-2v promoter (nucleotides −264 to +17, Genebank Accession No. U26708) may be used. This 281 bp fragment confers tissue-specificity when placed in a rAAV vector in vivo and when placed within a suitable vector in vitro (Phillips et al., Hypertension 39(2):651-655, 2002) Examples of other cardiac-specific promoters include alpha myosin heavy chain (Minamino et al., Circ. Res. 88:587-592, 2001) and myosin light chain-2 (Franz et al., Circ. Res. 73:629-638, 1993).

In other applications, promoters that confer gene expression specific to tissues other than the heart may be used. Promoters that are kidney-specific include CLCN5 (Tanaka et al., Genomics 58:281-292, 1999), renin (Sinn et al., Physical Genomics 3:25-31, 2000), androgen-regulated protein, sodium-phosphate cotransporter, renal cytochrome P-450, parathyroid hormone receptor and kidney-specific cadherin. See Am. J Physiol. Renal Physiol. 279:F383-392, 2000. The E-cadherin promoter directs expression specific to epithelial cells (Behrens et al., Proc Nat'l Acad. Sci. USA 88:11495-11499, 1991) while the Estrogen receptor (ER)₃ gene promoter directs expression specifically to the breast epithelium (Hopp et al., J. Mammary Gland Biol. Neoplasia 3:73-83, 1998). Endothelial cell gene promoters include endoglin and ICAM-2. See Velasco et al., Gene Ther. 8:897-904, 2001. Liver-specific promoters include the human phenylalanine hydroxylase (PAH) gene promoters (Bristeau et al., Gene 274:283-291, 2001), hB1F (Zhang et al., Gene 273:239-249, 2001), and the human C-reactive protein (CRP) gene promoter (Ruther et al., Oncogene 8:87-93, 1993). An example of a muscle-specific gene promoter is human enolase (ENO3). Peshavaria et al., Biochem. J. 292(Pt 3):701-704, 1993. A number of brain-specific promoters may be useful in the invention and include the thy-1 antigen and gamma-enolase promoters (Vibert et al., Eur. J. Biochem. 181:33-39, 1989), the glial-specific glial fibrillary acidic protein (GFAP) gene promoter (Cortez et al., J. Neurosci. Res. 59:39-46, 2000), and the human FGF1 gene promoter (Chiu et al., Oncogene 19:6229-6239, 2000). The GATA family of transcription factors have promoters directing neuronal and thymocyte-specific expression. See Asnagli et al., J. Immunol. 168:4268-4271, 2002. An example of a pancreas-specific promoter is the pancreas duodenum homeobox 1 (PDX-1) promoter. Samara et al., Mol. Cell Biol. 22:4702-4713, 2002.

### Other Regulatory Sequences

Examples of other regulatory sequences that may be used in vectors of the invention include insulators, silencers, enhancers, initiation sites, termination signals, internal ribosome entry sites (IRESs) and polyA tails. An example of a cardiac-specific enhancer is alpha BE-4 (Gopal-Srivastava et al., 15:7081-7090, 1995).

### Gene Amplification System

It is often the case that gene therapy protocols that utilize tissue-specific promoters fail to express sufficient quantities of a heterologous gene to achieve a therapeutic outcome. This is so because most known tissue-specific promoters are relatively weak promoters (compare the non-tissue-specific CMV promoter for instance). To overcome this limitation, the vector system of the invention utilizes a gene amplification system. This system includes the use of two components, a transactivator operably linked to a tissue-specific promoter and a regulatory sequence responsive to the transactivator (e.g., a transactivator-inducible promoter) operably linked to a heterologous gene. Activation of the tissue-specific promoter (which itself is generally a relatively weak promoter) causes expression of the transactivator. The transactivator, in turn, activates the transactivator-inducible promoter (which is generally a non-tissue specific, relatively strong promoter) to stimulate expression of the heterologous gene. For example, in the hypoxia-sensing, cardio-protective double AAV vector system described herein, the sensor vector encodes the GAL4-DBD/ODD/p65 AD transactivator. When present in combination with a reporter vector containing a yeast GAL4 UAS and an Ad E1b TATA element (e.g., one that includes six copies of a 17 bp GAL4 UAS and an Ad E1b TATA element) under hypoxic conditions, the transactivator fusion protein binds to the GAL4 UAS in the reporter vector and in conjunction with p65 AD vigorously stimulates high expression levels of the heterologous gene.

The gene amplification system can be used to induce expression of more than one type of heterlogous gene (e.g., 2, 3, 4, 5, or more). For example, the system can includes a transactivator operably linked to a tissue-specific promoter on a first vector, a regulatory sequence responsive to the transactivator (e.g., a transactivator-inducible promoter) operably linked to a first heterologous gene on a second vector, and a regulatory sequence responsive to the transactivator (e.g., a transactivator-inducible promoter) operably linked to a second heterologous gene on a third vector. Activation of the tissue-specific promoter (which itself is generally a relatively weak promoter) causes expression of the transactivator. The transactivator, in turn, activates the transactivator-inducible promoter on both the second and third vector to stimulate expression of both the first and second heterologous genes. By using additional constructs containing additional heterologous genes operably linked to a single type of transactivator-inducible promoter, the expression of any number of heterologous genes can be regulated by a single stimulus.

### Treatment of Various Disease States

The present invention further pertains to using the vector-based system for treating a variety of disease states in addition to cardiac ischemia. The two vector system can be adapted to a particular disease state by using an appropriate tissue-specific promoter, an appropriate gene switch/sensor element, and appropriate therapeutic genes for treating the particular disease state. For example, the stimulus-responsive vector system of the invention could be used to treat diabetes type 1 in a patient. For treating diabetes type 1, a transactivator vector contains a pancreas-specific promoter and a glucose sensitive element, for example, in addition to the GAL4 DBD and p65 AD sequences. A reporter vector to be used in combination with such a transactivator vector contains a pre-pro-insulin gene(s), for example, linked to the UAS and E1b TATA elements. Using this particular system, the transactivator vector detects elevated glucose levels and expresses a chimeric transactivator protein specifically in the pancreas. The transactivator protein then binds to the reporter vector and activates amplified expression of the pre-pro-insulin gene(s). When glucose levels are decreased the system switches off.

In another example, the stimulus-responsive vector system can be used to treat cancer. A transactivator vector for treating cancer, for example, may detect tumor markers and express a transactivator protein that activates anti-growth or anti-angiogenesis genes in the reporter vector. A further example of a disease state to be treated using the stimulus-responsive vector system is stroke. A transactivator vector responsive to hypoxia can express a transactivator protein that activates expression of the tPA gene in the reporter vector. In still another example, the stimulus-responsive vector system can be used to treat anthrax toxin where the protective gene is an anthrax toxin uptake receptor antisense molecule.

Many diseases are inflammatory diseases; for example arthritis, pulmonary fibrosis and atherosclerosis. A transactivator vector responsive to any of a number of atheroslcerosis indicators of inflammation including cytokines, MCP-1, c-reactive protein or elevated trigylceride, oxidised LDL cholesterol, Lp(a), homocysteine, and fibrinogen levels, as well as decreased HDL levels and endothelial-derived nitric oxide production, can be constructed. A transactivator protein expressed in response to such an indicator activates expression of a therapeutic gene in the reporter vector. Therapeutic genes for atherosclerosis include those that encode: 1) proteins with hypolipidemic activity, 2) proteins that act on the cholesteryl ester transfer protein and lipase systems, 3) cholesterol-removing proteins, 4) proteins with fibrinolytic activity, 5) proteins that provide low density lipoprotein receptor replacement, and/or 6) proteins that induce vascular protection and disobliteration of occlusions. Sirtori, C. R., Pharmacol. Ther. 67:433-47, 1995, 7) HO-1 and 8) LOX-1 antisense. Other disease states include anemia, the renal diseases that involve reduced oxygen. In the case of anemia, the therapeutic gene would be erythropoietin.

### Cardioprotective Genes

Within the invention are vectors that encode cardioprotective genes for protecting cells from ischemia in response to hypoxia. A number of antisense molecules that confer a cardioprotective effect are known, including antisense to angiotensin II type-1 receptor (Yang et al., Circulation 96:922-926, 1997; and Yang et al., Circ. Res. 83:552-559, 1998), antisense to adrenergic beta-1 receptor (Chen et al., Pharmacol. Exp. Ther. 294:722-727, 2000), and antisense to angiotensin-converting enzyme that has been shown to protect rat hearts from ischemia-reperfusion (Chen et al., Pharmacol. Exp. Ther. 294:722-727, 2000). Several cardioprotective genes are also known, including heme oxygenase-1. This protein degrades the pro-oxidant heme and generates carbon monoxide and antioxidant bilirubin, conferring myocardial protection from ischemia/reperfusion injury. Franz et al., Circ. Res. 73:629-638, 1993. In inflammatory diseases, HO-1 is increased as a cytoprotective gene. However, it is usually insufficient in amount to stop the inflammation. The vigilant vector could provide an amplified amount of HO-1 when reduced oxygen indicates a need for HO-1. Another example of a cardioprotective gene is superoxide dismutase, which protects heart tissue from super oxide radicals generated during ischemia-reperfusion. Chen et al., Circulation 94:II412-II417, 1996; and Woo et al., Circulation 98:II255-II260, 1998. Genes that provide a protective effect from other cardiac disease states, such as heart degeneration and failure, may also be used in vectors of the invention. An example of a gene that improves cardiac function is phospholanban (PLN). The PLN gene product regulates the strength of each heartbeat and is known to malfunction in heart failure. Zvaritch et al., J. Biol. Chem. 275:14985-14991, 2000. Any suitable cardioprotective gene that provides a therapeutically effective level of protection may be used within vectors of the invention.

### Administration of Compositions

The compositions described above may be administered to animals including human beings in any suitable formulation by any suitable method. For example, rAAV virions (i.e., particles) may be directly introduced into an animal, including by intravenous injection, intraperitoneal injection, or in situ injection into target tissue (e.g., cardiac tissue). For example, a conventional syringe and needle can be used to inject an rAAV virion suspension into an animal. Depending on the desired route of administration, injection can be in situ (i.e., to a particular tissue or location on a tissue), intramuscular, intravenous, intraperitoneal, or by another parenteral route. Parenteral administration of virions by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the rAAV virions may be in powder form (e.g., lyophilized) for constitution with a suitable vehicle, for example, sterile pyrogen-free water, before use.

To facilitate delivery of the rAAV virions to an animal, the virions of the invention can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly preferred for delivery of virions to human subjects. Methods for making such formulations are well known and can be found in, for example, Remington's Pharmaceutical Sciences.

In addition to the formulations described previously, the virions can also be formulated as a depot preparation. Such long acting formulations may be administered by implantation (for example subcutaneously or intramuscularly) or by IM injection. Thus, for example, the virions may be formulated with suitable polymeric or hydrophobic materials (for example as an emulsion in an acceptable oil) or ion exchange resins, or as sparingly soluble derivatives.

Similarly, rAAV vectors may be administered to an animal subject using a variety of methods. rAAV vectors may be directly introduced into an animal by peritoneal administration (e.g., intraperitoneal injection, oral administration), as well as parenteral administration (e.g., intravenous injection, intramuscular injection, and in situ injection into target tissue). Methods and formulations for parenteral administration described above for rAAV virions may be used to administer rAAV vectors.

Ex vivo delivery of cells transduced with rAAV vectors or infected with rAAV virions is also provided for within the invention. Cardiac myocyte-mediated ex vivo gene therapy may be used to transplant stimulus-responsive vector system-transduced (or cardioprotective gene product-containing) host cardiac myocytes back into the host heart. Similarly, ex vivo stem cell therapy may also be used to transplant stimulus-responsive vector system-transduced (or cardioprotective gene product-containing) host stem cells into the host heart. A suitable ex vivo protocol may include several steps. A segment of cardiac tissue may be harvested from the host and any suitable delivery vector may be used to transduce a stimulus-responsive rAAV vector system into the host's own cardiac cells. These genetically modified cells may then be transplanted back into the host. Several approaches may be used for the reintroduction of cardiac cells into the host, including jugular vein, tongue vein, and intracardiac intravenous delivery. Microencapsulation of cells transduced or infected with a stimulus-responsive rAAV vector system modified ex vivo is another technique that may be used within the invention. Autologous and allogeneic cell transplantation may be used according to the invention.

Any suitable delivery method may be used for transducing host stem cells or cardiac cells with a stimulus-responsive rAAV vector system ex vivo. Several suitable modes of delivery are described above and include the following: microinjection, electroporation, calcium phosphate transfection, DEAE dextran transfection, polylysine conjugates, receptor-mediated uptake systems, liposomes, lipid-mediated delivery systems, matrix-impregnated delivery, microparticle encapsulation, intra-cellular targeting ligands, virion-like particles, and viruses.

### Effective Doses

The compositions described above are preferably administered to a mammal in an effective amount, that is, an amount capable of producing a desirable result in a treated subject (e.g., protecting cells from ischemia in the subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population) and the ED₅₀ (the dose that produces the desired effect). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. It is expected that an appropriate dosage for intravenous administration of the compositions would be in the range of about 5 μl/kg at 10¹³ particles and 50 μl/kg at 10¹² particles. For a 70 kg human a 3 ml injection of 10¹² particles is presently believed to be an appropriate dose.

### EXAMPLES

The invention can be illustrated by the following examples in which a dual viral vector system is used to protect cells from ischemia-induced damage. In this application, an rAAV-based stimulus-responsive vector system activates expression of a trangene(s) in response to hypoxic conditions. This system utilizes a first rAAV vector (transactivator vector) having a cardiac-specific promoter linked to a sequence encoding an oxygen-sensitive chimeric transactivator, the RST. It contains a GAL4 DBD, an ODD and a p65 AD. ODD is a subunit of the HIF-1α protein, which under normal oxygen conditions is undetectable due to its rapid destruction by the ubiquitin-proteosome system. During hypoxia, however, HIF-1α is no longer degraded and accumulates exponentially as cellular O₂ decrease. The ODD controls ubiquitin-proteosome-mediated degradation of HIF-1α and confers oxygen-dependent instability when fused to a stable protein (e.g., GAL4).

This transactivator vector is administered in combination with a second rAAV vector that contains a cardioprotective gene linked to a GAL4 UAS sequence and TATA element. In response to hypoxia, the transactivator rAAV vector expresses the chimeric transactivator specifically in the heart. The transactivator binds to the GAL4 UAS sequence in the second rAAV vector, resulting in the expression of the cardioprotective gene. The examples described below are provided for illustration only and are not to be construed as limiting the scope or content of the invention in any way.

### Example 1

### Construction of Vectors and rAAV

rAAV: Methods to package rAAV for systemic injection have been described previously (Phillips M I. Hypertension. 1997;29:177-187.). A rat 1.7 kb MLC-2v promoter and coding sequence of green fluorescent protein (GFP) was inserted between the TRs of AAV vector to construct pMLC-2v-GFP (Phillips et al., Hypertension. 2002;39:651-655).

The MLC RST System includes a transactivator plasmid and a reporter plasmid: The transactivator plasmid (pGS-MLC-RST) is derived from pGS-CMV, which expresses a chimeric transcription factor, the RST, consisting of the yeast GAL4 DNA binding domain (DBD, amino acids 1-93) (Keegan, et al., Science 231, 699-704,1986.) and the human p65 activation domain (AD, amino acids 283-551) (Schmitz et al., EMBO J. 10, 3805-3817,1991) from NF-κB under the control of a CMV enhancer/promoter. The CMV promoter/enhancer in pGS-CMV was replaced by a 281 bp MLC fragment of pMLC-Luc amplified by (polymerase chain reaction) PCR to generate pGS-MLC. The ODD (amino acids 394-603) (L. E. Huang, et al., Proc.Natl.Acad.Sci. U.S.A 95, 7987-7992,1998) was amplified by PCR from pCEP4/HIF-1alpha (G. L. Semenza et al., J.Biol.Chem. 271, 32529-32537, 1996) and fused in frame between the coding sequence of GAL4 DBD and p65 AD in the pGS-MLC to generate pGS-MLC-RST. When inserted between GAL4 DBD and p65 AD, ODD conferred a powerful O₂ switch action. The reporter plasmid consisted of pGene-Lacz (Invitrogen, CA) which encodes β-galactosidase driven by six copies of a 17 bp GAL4 UAS (E. Giniger, S. et al., Cell 40, 767-774,1985) and an Ad E1b TATA box (J. W. Lillie et al., Nature 338, 39-44,1989). The lacZ coding sequence was replaced by firefly luciferase and secreted alkaline phosphatase (SEAP) cDNA to generate pGene-Luc and pGene-SEAP, respectively. pMLC-Luc: a 281 bp (−264 to +17, Genebank: U26708) fragment of MLC-2v-promoter was amplified by PCR from pMLC-2v-GFP (M. I. Phillips, et al., Hypertension 39, 651-655, 2002) and inserted into plasmid gene luciferase (pGL)-SV40 (Promega, Wis.) to replace SV40 promoter. The identity of clones was confirmed by nucleotide sequence analysis.

### Example 2

### Testing Vectors in Vitro

A rat embryonic cardiac myoblast cell line, H9c2 (ATCC: CRL 1446), was maintained in Dulbecco's modified Eagle's medium supplemented with sodium pyruvate and 10% fetal bovine serum. Cells were cultured under normoxic conditions (5% CO₂, 20% O₂, 75% N₂) in a humidified incubator at 37° C. For hypoxic treatment, cells were put into hypoxia chambers (oxygen sensors). The chambers were connected to a tank with pre-mixed gas (1% O₂, 5% CO₂, and 94% N₂ at the one end and to a vacuum at the other end. Hypoxia condition was achieved by evacuating and gassing the chambers six times. Then the tightly sealed chambers were incubated at 37° C. The oxygen level in the chamber and medium was around 7.6 mmHg, which was monitored by the OxyLite probe (Oxford Optronix, UK).

Transient transfection and reporter gene assays. Cells were transfected at a confluence of 50-60%. Transfection was performed with Lipofectamine (Invitrogen, Carlsbad, Calif.) according to manufacture's protocol. pRL-CMV (Promega, Madison, Wis.) or pRL-TK (Promega, Madison, Wis.) coding Renilla luciferase was used to control transfection efficiency. Luciferase assays were performed with dual luciferase assay system (Promega, Madison, Wis.). Results were quantified with a Monolight 3010 luminometer (Pharmingen, San Diego, Calif.), and expressed as a ratio of firefly luciferase activity over Renilla luciferase activity.

The pGL-MLC was specifically expressed in cardiomyocytes. After transient transfection, the luciferase expression in cardiomyocytes (H9c2) was 29.38+/−13.11-fold higher than that in rat glioma cells (C6) (Phillips et al., Hypertension. 39:651-655, 2002) and 20.07+/−1.71-fold higher than that in pulmonary vein endothelial cells (MPV). The transfection efficiency in three cell lines was normalized by the expression of an internal control plasmid containing ubiquitous viral promoter (CMV or thymidine kinase promoter).

### Example 3

### Routes of Injection for System Delivery of AAV-Based Vectors

Several routes of injection can be used for injecting the stimulus-responsive vectors and rAAV in vivo. Suitable protocols for use with rodents are described below. These can be adapted for other animal subjects according to standard veterinary and medical procedures.

Jugular vein. With the mouse in supine position, the jugular vein can be detected just under the neck skin by pulsation accompanying the respiratory rhythm. Using a 30 gauge needle, the vector is injected slowly in a volume of 50 μl. The advantage of the jugular route is that it has a large capacity for relatively large volumes and can be done without surgery.

Tongue vein injection. Under light anesthesia, the tongue is pulled forward, revealing the tongue vein. Again, using a 30 gauge needle, the vector is injected slowly in a volume of no more than 50 μl. The advantage of this method is that the vein is accessible without opening the skin, and the injectate can be seen entering the circulation.

Intracardiac injection. For intracardiac injection the animal is lightly anesthetized with an inhalant such as metofan or isoflurene and placed on its back. The needle of the syringe is injected under the xiaphoid process angle slightly left to enter the left ventricular chamber. The syringe is drawn back until blood is seen and then the injectate is injected directly into the blood. The advantage of this method is that it provides direct injections that are rapidly distributed through the body. In the course of experiments using this method of injection (Kimura et al., Hypertension 37(2):376-380, 2001), no toxic response or infection was observed.

Transfecting bone marrow stem cells. Bone marrow cells are removed with 19 g needle. The cells are cloned and transfected with the vigilant vector. These stem cells are then reinjected i.v. They will return to bone marrow but be released.

Direction injection into tissue. This approach has been used for injecting into heart, but requires general surgery, and therefore is not preferred. However, if the vigilant vector is designed to protect against arthritis of joints, the vector could be directly injected into joints under local anesthesia.

### Example 4

### Testing Cardiac Specificity In Vivo

Four weeks after a systemic injection into adult mouse or five-day-old rats, the transduction of rAAV-MLC-2v-GFP was shown by PCR of DNA in many tissues, such as spleen, liver, lung, kidney and heart. The tissue-specific expression of GFP MRNA under a MLC-2v promoter was examined by RT-PCR and was detected only in heart (Phillips et al., Hypertension 39:651-655, 2002). The presence of GFP protein was further examined by immunofluorescence staining, which was apparent in the heart of the treated animal and absent in the control animal (injected with saline). GFP was undetectable in the kidney and liver of the same treated animals and controls (Phillips et al., Hypertension 2002;39:651-655).

### Example 5

### Hypoxia Inducibility of the RST

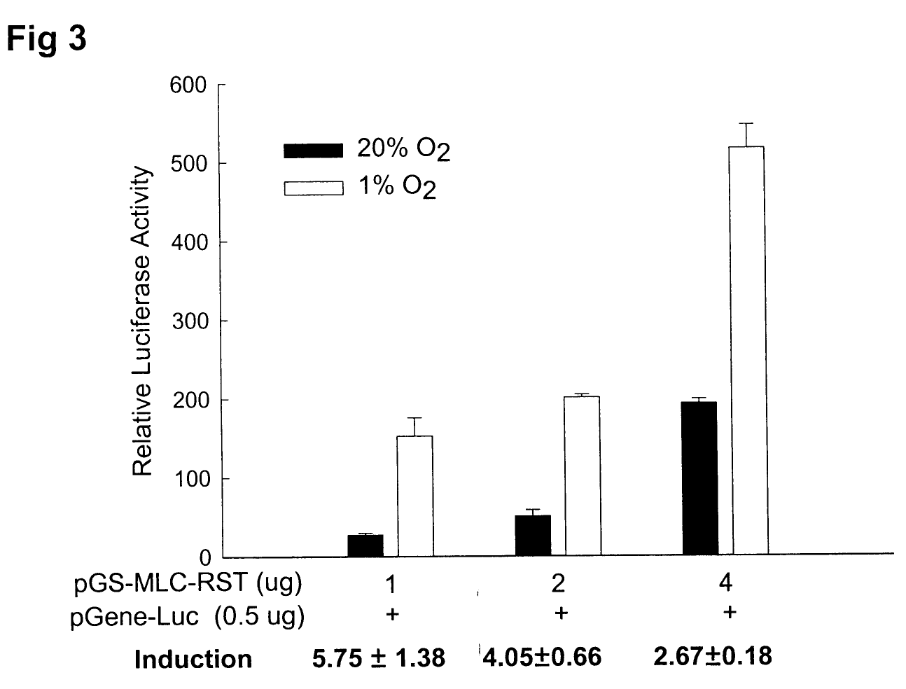

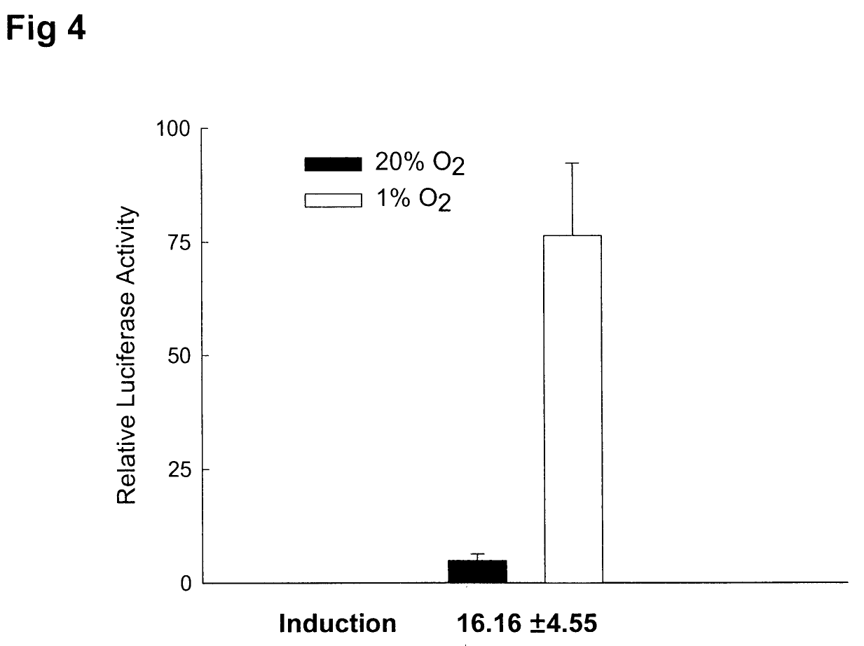

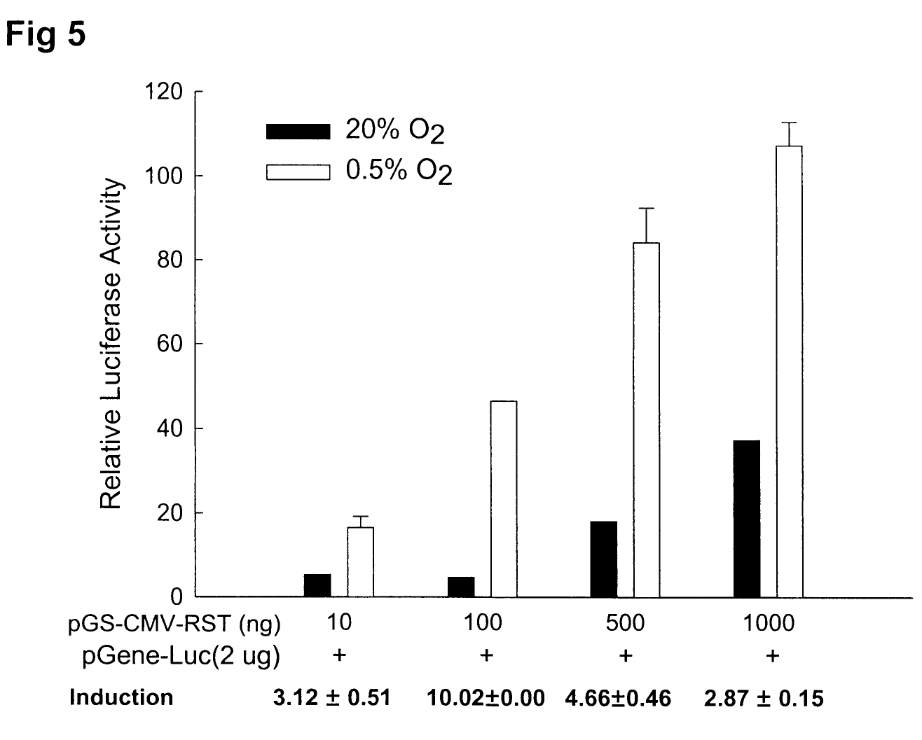

Increasing doses of transactivator plasmid (pGS-MLC-RST) and the same amount of reporter plasmid (pGene-Luc) were co-transfected into cardiomyocyte derived cells (H9c2). Then the cells were incubated at either 1% O₂ or 20% O₂ for 24 hours before preparing cell lysates (FIG. 3). The luciferase expression in the hypoxic cells increased 3 to 6-fold compared to that in the cells under nornoxia. Since H9c2 is an embryonic cell line, it may not represent the situation in the adult cardiomyocytes. Therefore, the finction of RST was tested in the primary culture of adult rat cardiomyocytes. One microgram per well of pGS-MLC-RST was transfected into the primary culture of adult rat cardiomyocytes along with 0.5 μg/well pGene-Luc and 20 ng/ well pRL-CMV control plasmid in 60 mm dishes. Triple plates were incubated at either 20% O₂ or 1% O₂ for 24 h before preparation of cell lysates. There was 16.16±4.55-fold hypoxia induction (FIG. 4). The RST has also been tested in human cells (FIG. 5). Human embryonic kidney epithelial cells (293) were co-transfected with 1 μg/well pRL-TK, 2 μg/well reporter plasmid (pGene-Luc), 10 ng-1000 ng/well transactivator plasmid (pGS-CMV-RST), and various amounts of empty vector such that all cells received a total of 4 μg of plasmid DNA. 24 h after transfection, cells were incubated at 0.5% or 20% O₂ for 24 h prior to preparation of cell lysates. There were 3 to 10-fold hypoxia inductions (FIG. 5). However, without ODD, the GAL4 DBD/p65 AD fusion protein did not significantly increase reporter expression.

### Example 6

### Time of Onset

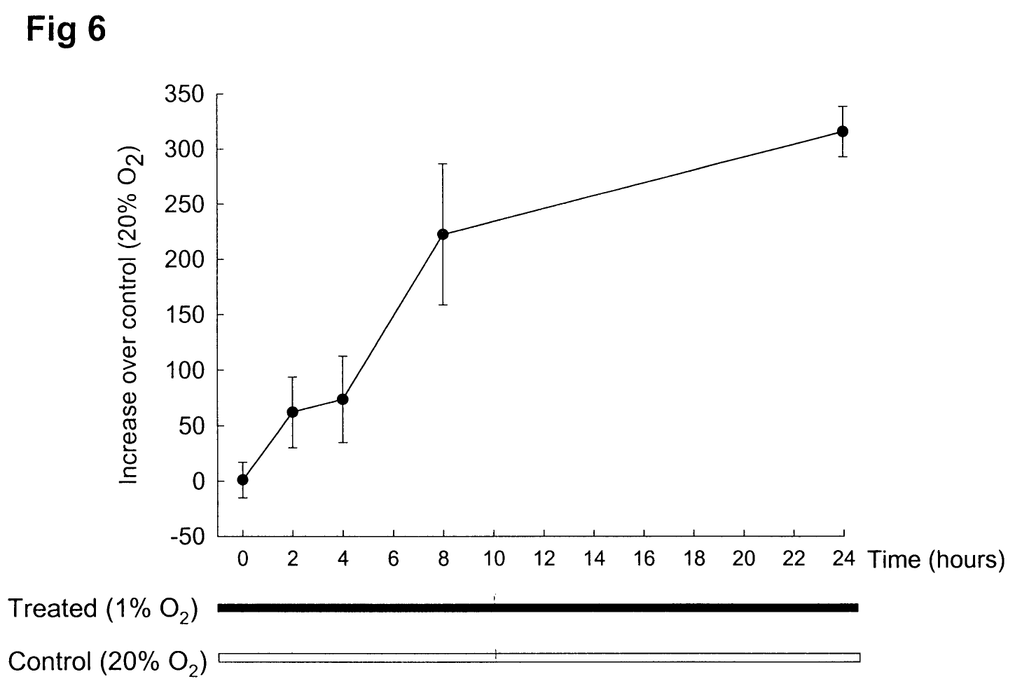

The time of onset in vitro was tested. H9c2 cells were transfected with 1 μg/well of pGS-MLC-RST, 0.5 μg/well pGene-Luc and 20 ng/ well pRL-CMV control plasmid, then were exposed to 1% or 20% O₂ for 0 to 24 hours. The cells were retrieved from different time points for luciferase assay. Measurements of O₂ levels in the cultures revealed that it took 2.6 to 3.8 hrs after O₂ had been reduced from 20% to 1% in a closed chamber to reach 1% in the culture medium. The reporter gene vector in this cell culture system was significantly higher at 2-6 hrs (FIG. 6). Therefore, subtracting the time to reach 1% in the media, the activation of the system was about 0.2 to 1.4 hrs. The time for therapeutic genes to act is within this time frame except in absolute anoxia.

### Example 7

### The ON and OFF Switch Function

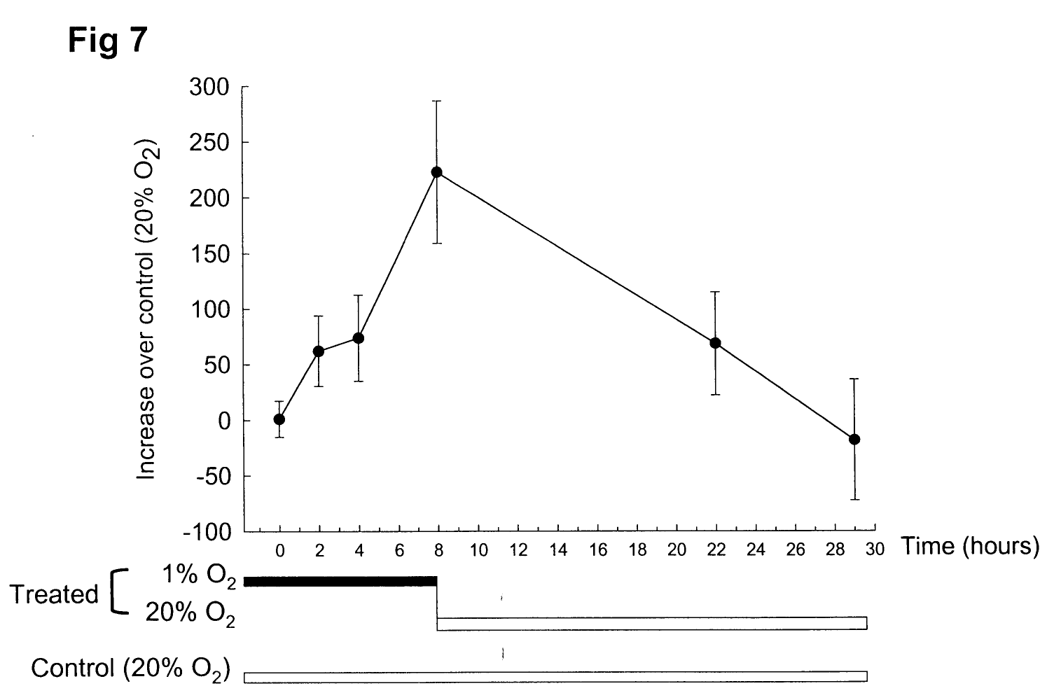

To be functional as a gene switch, the RST should be able to be turned on and off by the change of oxygen level. In order to test the switch on-and-off function of RST, the transfected H9c2 cells in treated group were first exposed at 1% O₂ for 8 h and then to 20% O₂ for another 21 hours (FIG. 7). H9c2 cells were transfected with 1 μg/well of pGS-MLC-RST, 0.5 μg/well pGene-Luc and 20 ng/ well pRL-CMV control plasmid. The transfected cells in the treated group were first exposed at 1% O₂ for 8 h then return to 20% O₂ for another 21 h. The cells in the control group were continuously incubated at 20% O₂. The cells in the control group were continuously incubated at 20% O₂. The luciferase expression in the treated group increased during 8 hours of hypoxic induction, compared to control. After 8 hours, the O₂ level was switched to 20% and the luciferase expression declined. After 21 hours of re-oxygenation, there was no difference in luciferase expression between the treated group and control group. This indicates that the RST switches on and off the transgene expression in response to the change of environmental oxygen tension.

### Example 8

### Cardiac Specific Expression

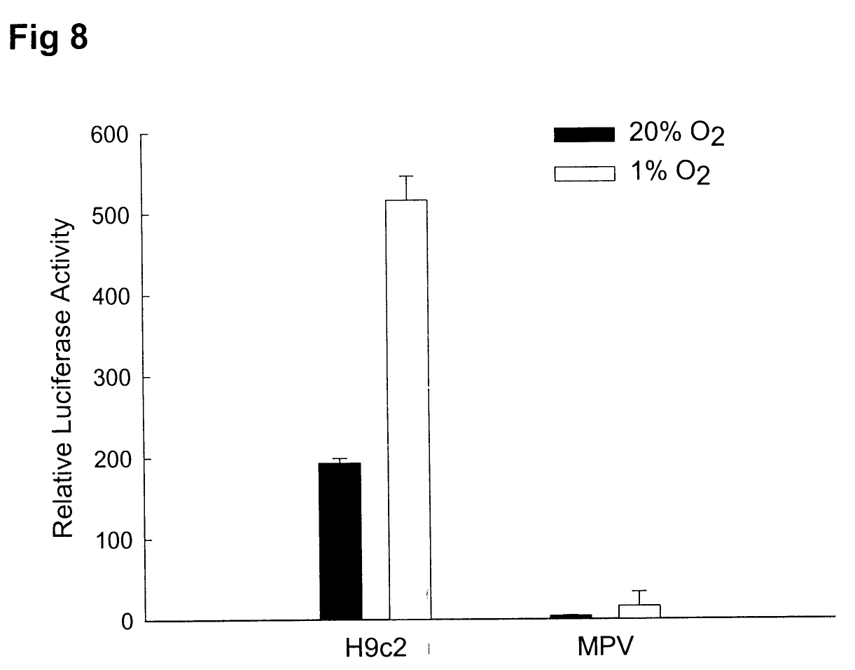

To verify if the control of transgene expression transferred from MLC-2v promoter by RST retains cardiac-specificity, the luciferase expression of MLC-RST system was also tested in non-cardiac cells (endothelial cells, MPV) under both hypoxic and normoxic conditions. The relative luciferase activity was tested in cardiac (H9c2) versus non-cardiac (MPV) cells which were transfected with 4 μg/well of pGS-MLC-RST, 0.5 μg/well pGene-Luc and 20 ng/well pRL-CMV control plasmid and exposed to 1% O₂ for 24 h. Myocardial cells specifically expressed the transgene under both normoxia and hypoxia condition. The expression level was very low in transfected MPV cells compared to that in the transfected cardiac myocytes (H9c2) in both normoxia and hypoxia (FIG. 8).

### Example 9

### Amplification Effect on Tissue Specific Promoter

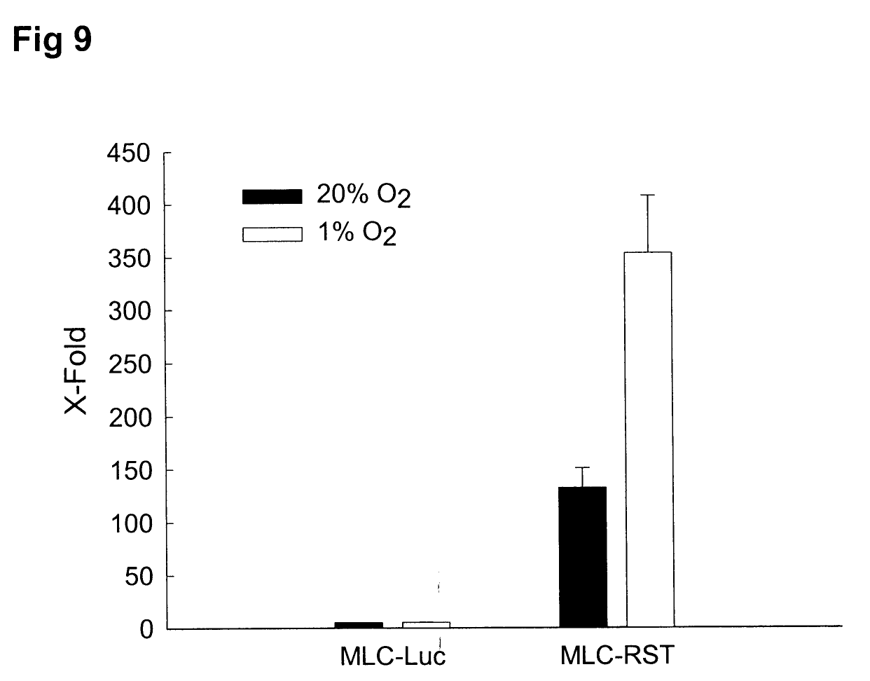

Since RST contains a strong transactivation domain from the p65, the ability of the RST to serve as an amplifier for the promoter was tested. The luciferase expression level of the MLC-RST system was compared to the expression from the plasmid (pMLC-Luc) in which the MLC-2v promoter drove the luciferase gene directly (FIG. 9). The RST amplifies the expression over 300-fold compared to a single plasmid system. In the MLC-RST group, the H9c2 cells were transfected with 4 μg/well of pGS-MLC-RST, 0.5 μg/well pGene-Luc and 20 ng/well pRL-CMV control plasmid. In the MLC-Luc group, H9c2 cells were transfected with 0.5 μg/well of pMLC-Luc, 20 ng/well pRL-CMV and 4 μg/well of empty plasmid. After transfection, cells were treated with either 1% O₂ or 20% O₂ for 24 h. The MLC-2v promoter alone could not be induced by hypoxia. The hypoxia induction ratio was 1.08±0.05. The MLC-RST system by contrast increased the reporter expression up to 353.36±54.28 fold under hypoxia. There was also an increase of 132.36±18.46 fold under normoxia relative to the expression of pMLC-Luc at 20% O₂.

### Example 10

### Single Regulator Switches on Multiple Reporter Genes

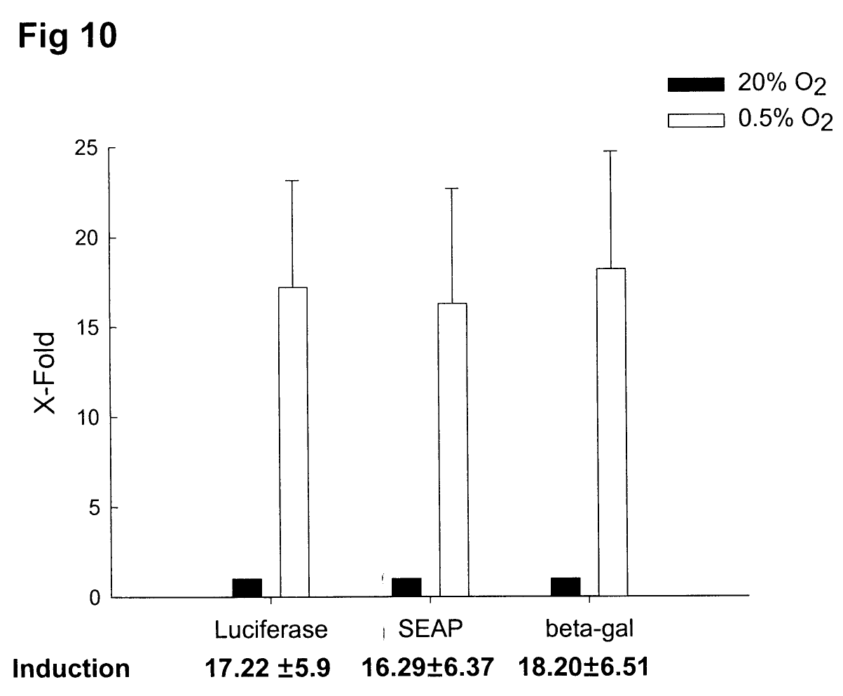

Since the RST fusion protein can recognize and activate GAL4 UAS, it should be possible to co-transfect multiple GAL4 UAS controlled transgenes with a single RST cassette and switch on all of the genes at the same time during hypoxia. To test this, one transactivator plasmid (pGS-MLC-RST) was cotransfected with several reporter plasmids which coded luciferase (Luc), β-Gal, and SEAP, respectively, and the cells were tested at 20 or 0.5% O₂. One microgram per well of transactivator plasmid (pGS-MLC-RST) and 0.5 μg/well of three reporter plasmids (pGene-Luc, pGene-secreted alkaline phosphatase (SEAP) and pGene-Lacz, respectively) were co-transfected into H9c2 cells. Twenty-four hours after transfection, the cells were subjected to either 0.5% or 20% O₂ for another 24 h before preparing cell lysate. The results are shown in FIG. 10. All three reporter genes showed a significant increase in their expression under hypoxia. This demonstrates that the RST system can enable multiple simultaneously in response to hypoxia.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for expressing a heterologous gene in a cell type-specific and inducible manner, the system comprising at least one vector comprising a nucleic acid encoding a switch/biosensor, a nucleic acid encoding a tissue-specific promoter, a nucleic acid encoding the heterologous gene, and a nucleic acid encoding a gene amplification component, wherein the switch/biosensor allows the system to regulate expression of the heterologous gene in response to a stimulus, the tissue-specific promoter allows the system to selectively express the heterologous gene in a particular cell type, and the gene amplification component induces expression of the heterologous gene at a level sufficient to exert a detectable physiological effect on a cell administered the system.

2. The system of claim 1, wherein the at least one vector comprises an rAAV vector.

3. The system of claim 1, wherein the at least one vector comprises at least a first rAAV vector and a second rAAV vector.

4. The system of claim 3, wherein the first rAAV vector comprises the nucleic acid encoding the switch/biosensor.

5. The system of claim 4, wherein the switch/biosensor comprises an oxygen-dependent degradation domain.

6. The system of claim 3, wherein the first rAAV vector comprises the nucleic acid encoding the tissue-specific promoter.

7. The system of claim 6, wherein the tissue-specific promoter comprises a cardiac-specific promoter.

8. The system of claim 7, wherein the cardiac-specific promoter is MLC-2v.

9. The system of claim 1, wherein the gene amplification component comprises a transactivator and a regulatory element responsive to the transactivator, wherein the regulatory element is operatively linked to the heterologous gene, and wherein binding of the transactivator to the regulatory element increases expression of the heterologous gene.

10. The system of claim 9, wherein the transactivator comprises a GAL4 DNA-binding domain.

11. The system of claim 10, wherein the transactivator further comprises a p65 activation domain.

12. The system of claim 10, wherein the regulatory element comprises a GAL4 UAS.

13. The system of claim 11, wherein the regulatory element comprises a GAL4 UAS and an Ad E1b TATA element.

14. The system of claim 9, wherein the switch/biosensor comprises an oxygen-dependent degradation domain, the tissue-specific promoter comprises a cardiac-specific promoter, the transactivator comprises a GAL4 DNA-binding domain and a p65 activation domain, the regulatory element comprises a GAL4 UAS and an Ad E1b TATA element.

15. The system of claim 14, wherein the cardiac-specific promoter is MLC-2v.

16. The system of claim 14, wherein the switch/biosensor, the tissue-specific promoter, and transactivator are encoded by nucleic acids comprised on a first rAAV vector, and the regulatory element and the heterologous gene are encoded by nucleic acids comprised on a second rAAV vector.

17. The system of claim 16, wherein the system comprises a third rAAV vector that comprises the regulatory element operably linked to a transgene differing from the heterologous gene.

18. The system of claim 1, wherein the system is comprised in at least one cell.

19. The system of claim 18, wherein the cell is in an animal subject.

20. A method for expressing a heterologous gene in a cell in a cell type-specific and inducible manner, the method comprising administering to the cell at least one vector comprising a nucleic acid encoding a switch/biosensor, a nucleic acid encoding a tissue-specific promoter, a nucleic acid encoding the heterologous gene, and a nucleic acid encoding a gene amplification component, wherein the switch/biosensor allows the system to regulate expression of the heterologous gene in response to a stimulus, the tissue-specific promoter allows the system to selectively express the heterologous gene in a particular cell or tissue type, and the gene amplification component induces expression of the heterologous gene at a level sufficient to exert a physiological effect on a cell administered the system.

21. The method of claim 20, wherein the cell is a cardiac myocyte.

22. The method of claim 20, wherein the cell is in an animal subject.
